(19) United States
(12) Patent Application Publication
(10) Pub. No.: US 2023/0142062 A1
(43) Pub. Date: May 11, 2023

(54) THE APPLICATION OF ALOESIN IN THE PREPARATION OF PRODUCTS FOR PREVENTING OR TREATING OBESITY

(71) Applicant: HAINAN MEDICAL UNIVERSITY, Haikou City, Hainan Province (CN)

(72) Inventors: Yan WANG, Haikou City, Hainan Province (CN); Youbin LI, Haikou City, Hainan Province (CN); Yinfeng TAN, Haikou City, Hainan Province (CN); Xuesong WANG, Haikou City, Hainan Province (CN); Junyu XU, Haikou City, Hainan Province (CN); Tingting ZENG, Haikou City, Hainan Province (CN); Xue CUI, Haikou City, Hainan Province (CN); Yu CHEN, Haikou City, Hainan Province (CN); Xueying ZHANG, Haikou City, Hainan Province (CN)

(21) Appl. No.: 17/605,851

(22) PCT Filed: Mar. 22, 2021
(86) PCT No.: PCT/CN2021/082124

§ 371 Date: Oct. 22, 2021

(30) Foreign Application Priority Data
Dec. 24, 2020 (CN) 202011543880.9

(51) Int. Cl. A61K 31/7048

(52) U.S. Cl. CPC A61K 31/7048

(57) ABSTRACT

In the technical field of plant medicines, disclosed is the application of aloesin in the preparation of products for preventing or treating obesity. Research has found that aloesin can inhibit lipid accumulation, increase the expression of browning genes UCP1 and PRDM16 and transcription factor C-EBPa, increase the expression and release of specific factor adiponectin, and inhibit the release of non-specific factor IL-1β, indicating that aloesin can mediate the browning of 3T3-L1 adipocytes, and aloesin can be used to prepare products for preventing or treating obesity.

### REFERENCE TO AN ELECTRONIC SEQUENCE LISTING

The contents of the electronic sequence listing (8629-0004 Sequence Listing.txt; Size: 3,318 bytes; and Date of Creation: Dec. 6, 2022) is herein incorporated by reference in its entirety.

### FIELD OF THE INVENTION

The invention relates to the technical field of plant medicines, in particular to the application of aloesin in the preparation of products for preventing or treating obesity.

### BACKGROUND OF THE INVENTION

Obesity refers to the accumulation of excess fat caused by calorie intake greater than consumption, and is currently one of the main health problems facing the world. In 2015, an estimated 609 million adults worldwide were obese, twice as many as in 1980. Obesity is a serious risk factor leading to insulin resistance, diabetes, atherosclerosis and cardiovascular disease. Changing dietary habits and increasing physical exercise are traditional methods of treating obesity. However, after controlling diet and increasing exercise, the fat reduction effect is temporary, and body weight will rebound. In addition, other methods such as bariatric surgery and medication also have their own risks and side effects such as alcoholism and acute kidney injury.

Aloe is a commonly used medicine in several traditional countries such as Greece, Egypt, India, Mexico, Japan and China. It is often used as a laxative to treat constipation in our country. Aloesin is an aromatic C-glycosylated 5-methylchromone compound, widely distributed in most aloe species and have a wide range of pharmacological effects. It is generally believed that it can directly inhibit the production of melanin and tyrosinase activity. In addition, aloesin can promote wound healing and improve diabetic syndrome by inhibiting oxidation and interfering with MAPK/Rho and Smad signaling pathways. Recently, new pharmacological effects of aloesin have been continuously reported, such as improving insulin resistance, inhibiting exacerbation of colitis in rats, inhibiting cell growth and metastasis of ovarian cancer SKOV3 cells, and inhibiting colorectal cancer cell growth by interfering with Wnt/β-catenin and Notch signaling pathways. However, whether aloesin has anti-obesity effects remains unclear.

### BRIEF SUMMARY OF THE INVENTION hi view of this, the present invention provides the application of aloesin in the preparation of anti-obesity agents, obesity treatment agents and related products.

Application of aloesin in the preparation of the anti-obesity agent for preventing obesity.

The application of aloesin in the preparation of an obesity therapeutic agent for treating obesity.

The application of aloesin in the preparation of a UCP1 gene expression promoter for preventing or treating obesity.

The application of aloesin in the preparation of a PDRM16 gene expression promoter for preventing or treating obesity.

The application of aloesin in the preparation of the UCP-1 and the PDRM16 gene expression promoters for preventing or treating obesity.

The application of aloesin in the preparation of a transcription factor C-EBPa expression promoter for preventing or treating obesity.

Application of aloesin in the preparation of an adiponectin accelerator for preventing or treating obesity.

Application of aloein in the preparation of a IL-1β inhibitor for preventing or treating obesity.

Compared with the prior art, the beneficial effects of the present invention are: the research of the present invention found that aloesin can inhibit lipid accumulation, increase the expression of browning genes UCP1 and PRDM16 and transcription factor C-EBPa, and improve the expression and release of specific factor adiponectin, inhibit the release of non-specific factor IL-1β, indicating that aloesin can mediate the browning of 3T3-L1 adipocytes, and aloesin can be used to prepare products for preventing or treating obesity. Moreover, aloesin is derived from plants and has high safety, providing a better choice for obese patients, and at the same time, making aloesin to play a higher value.

### BRIEF DESCRIPTION OF THE FIGURES

FIG. 1 shows the chemical structure (A) and experimental design (B) of aloesin. The effect of aloesin on cell viability after the pretreatment therewith for 24 h (C) and 48 h (D). n=6-12.

FIG. 2 shows the effect of aloesin on the differentiation of preadipocytes. Oil red O staining image (A), the lipid accumulation after the pretreatment with aloesin for 24 h (B), the lipid accumulation after the pretreatment with aloesin for 48 h, n=6 (C).

FIG. 3 shows the effect of aloesin on lipid accumulation in adipocytes. Oil red O stained image (A). Lipid accumulation of adipocytes treated with aloesin for 24 hours (B). Lipid accumulation of adipocytes treated with aloesin for 48 h, n=6(C).

FIG. 4 show the effect of aloesin on the mRNA expression of UCP-1, PRDM16, PPAR-γ, C-EBPa and FABP in 3T3-L1 adipocytes. n=4-8.

FIG. 5 shows the effect of aloesin on the mRNA expression and release of adiponectin and IL-1β in 3T3-L1 adipocytes. n=3-8.

### DETAILED DESCRIPTION OF THE INVENTION

In order to better understand the technical content of the present invention, specific embodiments are provided below to further illustrate the present invention.

Unless otherwise specified, the experimental methods used in the embodiments of the present invention are conventional methods.

The materials, reagents, etc. used in the embodiments of the present invention can be obtained from commercial sources unless otherwise specified.

3T3-L1 preadipocytes were originally derived from Swiss mouse embryonic tissue. Because of its potential to differentiate from fibroblasts into mature adipocytes, this cell line has been widely used in the study of mechanisms of obesity, diabetes and related diseases. Therefore, in this experiment, we will select 3T3-L1 pre-adipocytes and adipocytes to explore the effect of aloesin on adipocytes.

1. Materials and Methods

1.1 Cell Culture

Mouse 3T3-L1 preadipocytes were inoculated with high glucose culture medium (H-DMEM, Hyclon) containing 10% fetal bovine serum (Gibco) and antibiotics (100 U/mL penicillin G and 100 mg/mL streptomycin) at a density of 1×10⁴ /mL in an incubator at 37° C. and 5% CO₂ for 72 hours. In order to induce the differentiation of 3T3-L1 preadipocytes, when 80% cells are fused, on day 0, growth medium (GM) containing 10% fetal bovine serum, 10 μg/mL insulin (sigma), 1 μM dexamethasone (sigma), 0.5 mM isobutyl-1-Methylxanthine (sigma) was added therein. On the second day, the medium was replaced with a high-glycemic medium containing 10% calf serum and 10 μg/mL insulin. On the 10th day, a large number of lipid droplets accumulated during the differentiation of pre-adipocytes.

1.2 MTT Method

Cell viability was determined by MTT, 3T3-L1 preadipocytes were seeded in 96-well plates at a density of 1×10⁴ /mL, cultured in an incubator for 24 hours, and then replaced with a medium respectively containing 10, 15, 30 and 60 μM aloesin to continuously cultivate for 24 h or 48 h. 0.5% MTT was added to the cells, after incubating for 1 hour at 37° C., the MTT solution was removed, and DMSO was added to dissolve. The absorbance at 570 nm was measured using an enzyme-labeled instrument (Molecular Devices, CA, USA). Cell viability (%) is expressed as a percentage relative to untreated control cells.

1.3 Oil Red Staining

The differentiated adipocytes were washed 3 times with PBS, fixed with 10% formaldehyde for 1 hour, then washed 3 times with PBS again, dried for 15 to 30 minutes and stained with Oil Red O working solution for 2 hours. Wash 2 times and 3 times with 60% isopropanol (IPA) and double distilled water respectively. A Zeiss fluorescence microscope (Tokyo, Japan) was used to collect images. 100% IPA extracts Oil Red O, and the absorbance at 490 nm was measured.

1.4 Real-Time Quantitative PCR Analysis

Total RNA extraction reagent (TIANGEN, China) was used to extract total RNA from 3T3-L1 adipocytes. Use 1^{st} strand cDNA kit (Vazyme biotechnology) for reverse transcription into cDNA. SYBR Green PCR Master Mix Kit (Monad, China) reagent and CFX-1000 Touch instrument (Applied Biosystems, USA) were used to detect the mRNA level of adipocyte browning genes (Ucp-1 and PRDM16) and transcription factors (Fabp, Ppar-γ and C-EBPα). The following conditions were used for reverse transcription: 5 min at 25° C., 15 min at 50° C., and 5 min at 85° C. Amplification: initial denaturation at 95° C. for 30 seconds, followed by denaturation at 95° C. for 5 seconds and 59° C. for 30 seconds (40 cycles). The β-actin was used as a reference gene to standardize the qPCR reaction. The relative expression of genes was calculated based on the average Ct value between samples. Relative expression level=2\hat{ }{[Ct_{target gene}−Ct (β-actin)]_{aloesin treatment group}−[Ct_{target gene}−Ct(β-actin)] 3T3-L1_{adipocyte}]}.

TABLE 1
Primer sequence

| Gene | Forward primer | Reverse primer |
|---|---|---|
| name | (3′-5′) | (3′-5′) |
| β-actin | CCACAGCTGAG | AAGGAAGGCT |
| | AGGGAAATC | GGGAAAAGA |
| UCP-1 | CCTGCCTCTCT | GTAGCGGGGT |
| | CGGAAACAA | TTTGATCCCA |
| PRDM16 | CCCCACATTCC | CTCGCAATCCT |
| | GCTGTGAT | TGCACTCA |
| PPAR-γ | CAAGAATACC | GAGCTGGGTC |
| | AAAGTGCGAT | TTTTCAGAA |
| | CAA | TA ATAAG |
| C-EBPα | AGCAACGAGT | TGTTTGGCTTT |
| | ACCGGGTAC | ATCTCGGCTC |
| FABP | AGTGAAAATT | GCCTGCCACTT |
| | CGATGATTAC | TCCTTGTG |
| | ATGAA | |
| Adiponectin | CAGGTCTTCT | TCCACATTCTT |
| | TGGTCCTAAG | CTTCCTGATAT |
| | GG | G |
| IL-1β | TGCCACCTTTT | GGAGCCTGTAG |
| | AGACAGTGTG | TGCAGTTGT |

1.5 Enzyme-Linked Immunosorbent Assay (ELISA)

The 3T3-L1 adipocytes were treated with aloesin for 48 hours, then the supernatant was collected, and the levels of adiponectin and IL-1β in the supernatant were detected with an ELISA kit (Proteintech, China). The ELISA reagent operation was carried out according to the manufacturer's instructions.

1.6 Statistical Analysis

The result value is expressed as mean±standard error. Using GraphPad Prism 8 software to analyze statistical significance by one-way analysis of variance or two-sided T-test, P<0.05 is considered to be of statistic difference.

2. Results

2.1 The Effect of Aloesin on the Viability of 3T3-L1 Preadipocytes

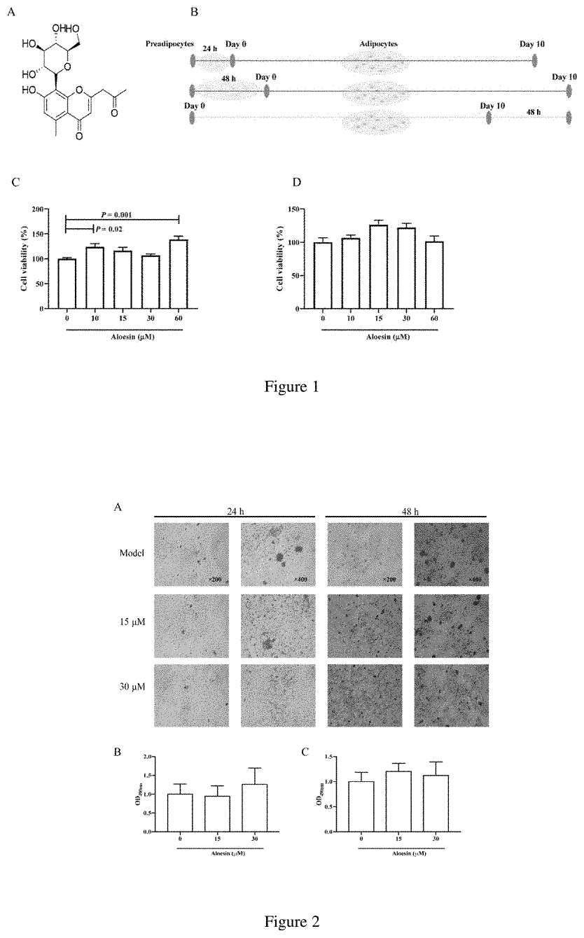

Effect of aloesin on the 3T3-L1 pre-adipocyte viability was evaluated by MTT assay. Pre-adipocytes were incubated with increasing concentration of aloesin (10 to 60 μM) for 24 h and 48 h. It was illustrated that pre-treated with aloesin (15 and 30 μM) for 24 h and 48 h did not influence the pre-adipocytes viability. Hence, the concentration of 15 and 30 μM was selected for further investigation (FIGS. 1C and 1D).

As shown in FIG. 1, the chemical structure (A) and experimental design (B) of aloesin. The effect on cell viability with aloesin pretreatment for 24 h (C) and 48 h (D). n=6-12.

2.2 Aloesin Does Not Affect the Differentiation of Preadipocytes

3T3-L1 pre-adipocytes were pre-incubated by the aloesin with concentration of 15 and 30 μM for 24 h and 48 h, respectively. Then pre-adipocytes were differentiated for 10 days. No influence was found after pretreated by 15 and 30 μM of aloesin for 24 h and 48 h (FIG. 2A and FIG. 2B) and 48 h (FIG. 2A and FIG. 2C) did not affect the differentiation of 3T3-L1 preadipocytes.

2.3 Aloesin Reduces Lipid Accumulation in 3T3-L1 Adipocytes

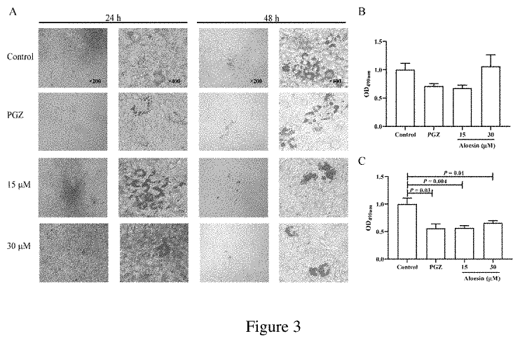

After 3T3-L1 preadipocytes were induced to differentiate into adipocytes, aloesin (15 and 30 μM) was added to the adipocytes and cultured for 24 h and 48 h respectively. The results showed that aloesin (15 and 30 μM) treatment for 24 hours did not affect the lipid accumulation in 3T3-L1 adipocytes (FIG. 3A and FIG. 3B). However, compared with the control group, treatment with 15 and 30 μM aloesin for 48 h could significantly reduce the lipid accumulation of adipocytes (FIG. 3A and FIG. 3C).

2.4 Aloesin Reduces Lipid Accumulation in Adipocytes by Inducing Browning

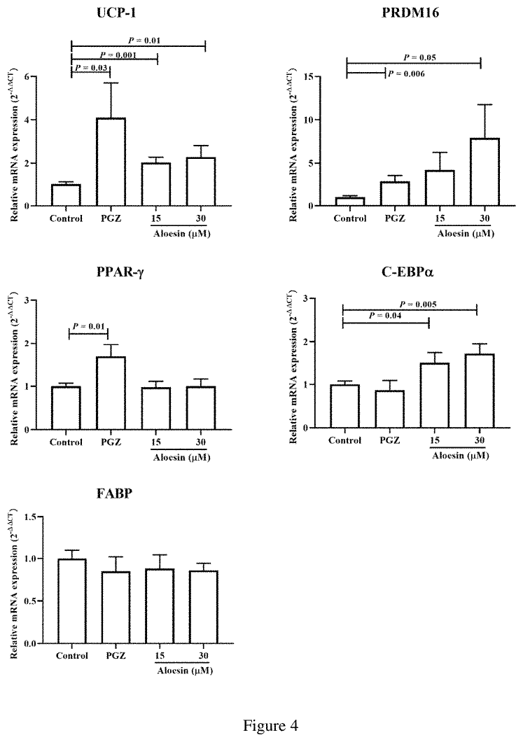

In order to further explore the mechanism of the influence of aloesin on lipid accumulation, we analyzed the mRNA expression of key genes related to adipocyte browning by real-time fluorescent quantitative PCR. It was found that aloesin could increase the expression of browning genes (UCP-1 and PDRM16). Further results showed that both 15 and 30 μM aloesin could increase the expression of the transcription factor C-EBPα, but did not affect FABP and PPAR-γ, indicating that the effect of aloesin on the transcription of UCP-1 and PDRM16 may be regulated by C-EBPα, but not related to FABP and PPAR-γ (FIG. 4).

2.5 Aloesin Increases the Release of Specific Cytokines (Adiponectin) in the Adipocyte and Reduces the Expression of Non-Specific Cytokines (IL-1β) Therein

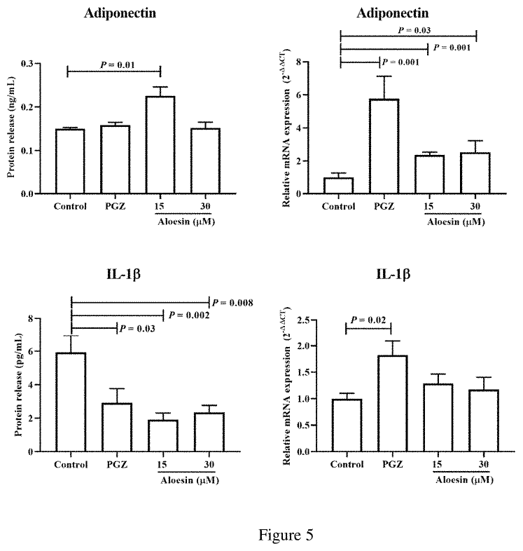

15 μM aloesin can enhance the mRNA expression and release of adiponectin in adipocytes, while 15 and 30 μM aloesin can reduce the release of IL-1β in adipocytes (FIG. 5).

In this study, it is found that (1) Aloesin can reduce lipid accumulation by activating the browning of white adipocytes, and improve the specific and non-specific pro-inflammatory effects of adipocytes.

(2) Aloesin can reduce lipid accumulation in 3T3-L1 adipocytes and increase the mRNA expression of UCP1 and PRDM16, so it is indicated that aloesin may inhibit lipid accumulation by inducing browning of adipocytes.

(3) Aloesin can increase the expression and secretion of adiponectin mRNA, reduce the secretion of non-specific cytokine IL-1β, and consolidate the effect of aloesin in inducing the browning of 3T3-L1 adipocytes.

(4) Aloesin can enhance the fat to form the adipogenic transcription factor C-EBPα, but not the mRNA expression of FABP and PPAR-γ, so it is indicated that the mechanism of aloesin inducing browning is related to its enhancement of the expression of the transcription factor C-EBPα.

In summary, the present invention indicates that aloesin can inhibit lipid accumulation, increase the expression of browning genes UCP1 and PRDM16 and the transcription factor C-EBPa, increase the expression and release of the specific factor adiponectin, and inhibit the release of the non-specific factor IL-1β, it is illustrated that aloesin can mediate the browning of 3T3-L1 adipocytes. Aloesin can be used in the preparation of products for preventing or treating obesity and can be used in the preparation of anti-obesity agents for preventing obesity or obesity treatment agents for treating obesity, etc.

The above are only the preferred embodiments of the present invention and are not intended to limit the present invention. Any modification, equivalent replacement, improvement, etc. made within the spirit and principle of the present invention shall be included in the scope of protection of the present invention.

1.-8. (canceled)

9. A composition comprising an anti-obesity agent capable of preventing obesity, wherein the anti-obesity agent comprises aloesin.

10. A composition comprising an obesity therapeutic agent capable of treating obesity, wherein the obesity therapeutic agent comprises aloesin.

11. A composition comprising a UCP1 gene expression promoter, a PDRM16 gene expression promoter, a transcription factor C-EBPa expression promoter, an adiponectin accelerator, and a IL-1β inhibitor capable of preventing or treating obesity, wherein the UCP1 gene expression promoter, the PDRM16 gene expression promoter, the transcription factor C-EBPa expression promoter, the adiponectin accelerator, and the IL-1β inhibitor comprise aloesin.

12. A method of preventing or treating obesity, comprising administering an effective dose of the composition of claim 9 to a patient in need thereof.

13. A method of preventing or treating obesity, comprising administering an effective dose of the composition of claim 10 to a patient in need thereof.

14. A method of preventing or treating obesity, comprising administering an effective dose of the composition of claim 11 to a patient in need thereof.


SEQUENCE LISTING (16 sequences)

SEQ ID NO: 1 - DNA, 20 bp, Artificial Sequence
  Beta-actin
ccacagctgagagggaaatc

SEQ ID NO: 2 - DNA, 19 bp, Artificial Sequence
  Beta-actin
aaggaaggctggaaaagag

SEQ ID NO: 3 - DNA, 20 bp, Artificial Sequence
  UCP1
cctgcctctctcggaaacaa

SEQ ID NO: 4 - DNA, 20 bp, Artificial Sequence
  UCP1
gtagcggggtttgatcccat

SEQ ID NO: 5 - DNA, 19 bp, Artificial Sequence
  PRDM16
ccccacattccgctgtgat

SEQ ID NO: 6 - DNA, 19 bp, Artificial Sequence
  PRDM16
ctcgcaatccttgcactca

SEQ ID NO: 7 - DNA, 23 bp, Artificial Sequence
  PPAR-gamma
caagaataccaaagtgcgatcaa

SEQ ID NO: 8 - DNA, 26 bp, Artificial Sequence
  PPAR-gamma
gagctgggtcttttcagaataataag

SEQ ID NO: 9 - DNA, 19 bp, Artificial Sequence
  CCAAT/enhancer-binding protein alpha
agcaacgagtaccgggtac

SEQ ID NO: 10 - DNA, 21 bp, Artificial Sequence
  CCAAT/enhancer-binding protein alpha
tgtttggctttatctcggctc

SEQ ID NO: 11 - DNA, 25 bp, Artificial Sequence
  FABP
agtgaaaattcgatgattacatgaa

SEQ ID NO: 12 - DNA, 19 bp, Artificial Sequence
  FABP
gcctgccactttccttgtg

SEQ ID NO: 13 - DNA, 22 bp, Artificial Sequence
  Adiponectin
caggtcttcttggtcctaaggg

SEQ ID NO: 14 - DNA, 23 bp, Artificial Sequence
  Adiponectin
tccacattcttttcctgatactg

SEQ ID NO: 15 - DNA, 21 bp, Artificial Sequence
  Interleukin-1 beta
tgccaccttttgacagtgatg

SEQ ID NO: 16 - DNA, 20 bp, Artificial Sequence
  Interleukin-1 beta
ggagcctgtagtgcagttgt